(19) United States
(12) Patent Application Publication
(10) Pub. No.: US 2011/0190834 A1
(43) Pub. Date: Aug. 4, 2011

(54) DEVICES AND METHODS FOR SUPPRESSION OF SYMPATHOEXCITATION

(76) Inventor: Jeffrey Goldberger, Skokie, IL (US)

(21) Appl. No.: 13/018,956

(22) Filed: Feb. 1, 2011

Related U.S. Application Data
(60) Provisional application No. 61/301,079, filed on Feb. 3, 2010.

(51) Int. Cl. A61N 1/362; A61N 1/39

(52) U.S. Cl. 607/5; 607/9; 607/14

(57) ABSTRACT

The present invention relates to devices and methods for suppression of sympathoexcitation and/or sudden cardiac death. In particular the present invention provides devices and methods which prevent sympathoexcitation and/or sudden cardiac death through an elevated heart rate stimulus.

### CROSS-REFERENCE TO RELATED APPLICATIONS

The present application claims priority to U.S. Provisional Patent Application Ser. No. 61/301,079, filed Feb. 3, 2010, which is herein incorporated by reference in its entirety.

### STATEMENT REGARDING FEDERALLY SPONSORED RESEARCH OR DEVELOPMENT

This invention was made with government support under grant number 1 R01 HL 070179-01A2 awarded by the National Institute of Health. The government has certain rights to the invention.

### FIELD OF THE INVENTION

The present invention relates to devices and methods for suppression of sympathoexcitation and/or sudden cardiac death. In particular the present invention provides devices and methods which prevent sympathoexcitation and/or sudden cardiac death through an elevated heart rate stimulus.

### BACKGROUND OF THE INVENTION

The “fight-or-flight” response is a critical human protective mechanism that is activated during a variety of stressors. Once activated, the sympathetic activation typically lasts far longer than the period of actual stress. While a prolonged duration for this response was teleologically an important adaptive response for our ancestors who faced physical dangers in their immediate surroundings that typically would not resolve quickly, the prolonged duration in modern day society could be maladaptive. Specifically, modern humans are typically exposed to brief stimuli that result in sympathoexcitation, but the effects may linger for long periods of time after the stimulus has resolved. As sympathoexcitation has been associated with ventricular tachyarrhythmias and sudden death, the sympathoexcitation that persists for extended periods of time when the stimulus has resolved may result in an increased risk of sudden death.

Exercise, the prototypical condition associated with sympathetic activation and parasympathetic withdrawal, and the post-exercise recovery period are associated with a dramatically increased risk for sudden death—up to 20-fold compared to sedentary periods (Whang et al. JAMA. 2006; 295: 1399, Albert et al. N Engl J. Med. 2000; 343: 1355, herein incorporated by reference in their entireties). This enhanced risk extends into the post-exercise recovery period and can be detected, even though regular exercise is known to provide overall health benefits including a reduced risk of sudden death—the “exercise paradox”. After brief periods of moderate exercise in normal subjects and those with coronary artery disease, there is evidence of sympathetic activation that persists for at least 45 minutes into the recovery period. Enhanced sympathetic activity may impair endothelial function of the brachial artery. Furthermore, analyses of the triggers for shocks from implantable defibrillators show that over half are related to exertion or other conditions of sympathoexcitation.

While sympathoexcitation is critical to support the body's physiologic demands during exercise, it is not clear that persistent sympathoexcitation is needed for extensive periods of time in recovery. While beta-adrenergic blockers are very effective agents to reduce the incidence of sudden cardiac death, because they are competitive blockers, sympathoexcitation during exercise and recovery is still detected in patients with coronary artery disease on beta-blocker therapy. To date, there are no selective therapies that can quickly “shut off” the body's sympathetic response once it has been activated, even if it is not needed.

### SUMMARY OF THE INVENTION

In some embodiments, the present invention provides a method of suppressing sympathoexcitation in a subject comprising administering cardiac pacing to the subject. In some embodiments, sympathoexcitation comprises post-exercise sympathoexcitation. In some embodiments, the cardiac pacing comprises and increased heart rate over the subject's resting heart rate. In some embodiments, the cardiac pacing is administered by a defibrillator or pacemaker. In some embodiments, the defibrillator comprises a cardioverter defibrillator. In some embodiments, suppressing sympathoexcitation reduces the subject's risk of ventricular tachyarrhythmia. In some embodiments, suppressing sympathoexcitation reduces the subject's risk of sudden cardiac death.

In some embodiments, the present invention provides a device comprising a sensor component and a heart rate regulation component, wherein activation of the sensor component causes the heart rate regulation component to raise a subject's bradycardia pacing rate. In some embodiments, raising the bradycardia pacing rate comprises raising the rate from the nominal pacing rate at rest conditions to a higher pacing rate. In some embodiments, the device is implantable. In some embodiments, raising the bradycardia pacing rate suppresses sympathoexcitation in a subject. In some embodiments, raising the bradycardia pacing rate reduces a subject's risk of ventricular tachyarrhythmia. In some embodiments, raising the bradycardia pacing rate reduces a subject's risk of sudden cardiac death. In some embodiments, the heart rate regulation component comprises a pacemaker or defibrillator.

### DESCRIPTION OF THE DRAWINGS

FIG. 1 shows graphs of norepinephrine and epinephrine levels versus time in the presence and absence of atropine.

FIG. 2 shows a graph of QTc interval (msec) versus time.

### DETAILED DESCRIPTION OF THE INVENTION

The present invention relates to devices and methods for suppression of sympathoexcitation and/or sudden cardiac death. In particular the present invention provides devices and methods which prevent sympathoexcitation and/or sudden cardiac death through an elevated heart rate stimulus.

Experiments conducted during development of embodiments of the present invention have demonstrated that after brief periods of moderate exercise in normal subjects and those with coronary artery disease, there is evidence of sympathetic activation that persists for at least 45 minutes into the recovery period. Enhanced sympathetic activity may impair endothelial function of the brachial artery (Lind et al. Blood Press. 2002; 11: 22, Hijmering et al. J Am Coll Cardiol. 2002; 39: 683, Joras et al. Vasc Med. 2008; 13: 255, herein incorporated by reference in their entireties). Furthermore, analyses of the triggers for shocks from implantable defibrillators show that over half are related to exertion or other conditions of sympathoexcitation.

While sympathoexcitation is critical to support the body's physiologic demands during exercise, it is not clear that persistent sympathoexcitation is needed for extensive periods of time in recovery. While beta-adrenergic blockers are very effective agents to reduce the incidence of sudden cardiac death, because they are competitive blockers, sympathoexcitation during exercise and recovery is still detected in patients with coronary artery disease on beta-blocker therapy. To date, there are no selective therapies that can quickly “shut off” the body's sympathetic response once it has been activated, even if it is not needed. Administration of atropine at the end of exercise results in a decline in plasma catecholamine levels, so that during recovery plasma catecholamine levels were slightly lower than baseline values. Muscarinic blockade increases norepinephrine release in the setting of nicotinic agonists (Lindmar et al. Br J Pharmac Chemother. 1968; 32: 280, herein incorporated by reference in its entirety). It is unlikely that atropine, a muscarinic blocker, exerts a direct effect to decrease plasma catecholamine levels. Another feedback loop is operative to reduce plasma catecholamine levels. Elevated heart rate, resulting from atropine administration, cardiac pacing, etc, results in a reflex suppression of sympathoexcitation.

Although the prognostic significance of heart rate is clear, some of the benefit of a lower heart rate is related to diminished sympathoexcitation, enhanced parasympathetic activity, and/or treatment with pharmacologic agents such as beta-blockers, rather than an isolated effect of heart rate. In patients with coronary artery disease, heart rate reduction may prevent ischemia and ischemic-related arrhythmias. The effect of heart rate on outcome has been extensively studied after myocardial infarction particularly in relation to drugs that reduce heart rate. Reduction in infarct size by acute administration of beta-blockers has been related to the degree of heart rate lowering. In long-term trials, the reduction in mortality and reinfarction was also related to the reduction in heart rate. Heart rate reduction represents a pharmacologic response to the administration of beta-blockers. However, other drugs that slow the heart rate have been evaluated as potential prophylactic agents for use in the post-myocardial infarction setting. Calcium channel blockers, such as diltiazem and verapamil that slow the heart rate have not proven to be beneficial in this setting. Amiodarone also lowers heart rate and has not been shown to have a beneficial effect on total mortality in the European (EMIAT) and Canadian (CAMIAT) post-myocardial infarction studies. Thus, rate slowing by itself is not a sufficient treatment goal to provide a survival advantage.

Clinical trial in patients with congestive heart failure demonstrated the survival benefits of beta-blockers are not dependent on the initial heart rate, nor on the extent of heart rate lowering achieved by beta-blocker treatment (Lechat et al. Circulation. 2001; 103: 1428, herein incorporated by reference in its entirety). Patients who had an increase in their heart rate on beta-blocker therapy experienced a survival benefit. MERIT-HF also noted no relation of the benefit of metoprolol based on initial or achieved heart rate (Gullestad et al. J Am Coll Cardiol. 2005; 45: 252, herein incorporated by reference in its entirety). These data indicate that heart rate reduction is not the mediator of all the clinical benefits of beta-blocker therapy following a myocardial infarction. Experimental and clinical studies have demonstrated that certain alterations in autonomic effects in the setting of structural cardiovascular disease are associated with an adverse prognosis. Both increased sympathetic and decreased parasympathetic activity have been associated with an increased risk for sudden death and/or susceptibility to ventricular arrhythmias. For example, there is an increased incidence of reperfusion-induced ventricular fibrillation with sympathetic stimulation in dogs (Miyazaki et al. Circulation. 1990; 82: 1008, herein incorporated by reference in its entirety). Conversely, parasympathetic stimulation decreases the incidence of ventricular fibrillation during ischemia in exercising dogs with myocardial infarctions (Vanoli et al. Circ Res. 1991; 68: 1471, herein incorporated by reference in its entirety). In humans, beta-blockers decrease the incidence of sudden cardiac death after myocardial infarction and in patients with congestive heart failure. The changes in autonomic effects associated with improved outcomes are precisely those that lower the heart rate, indicating that heart rate serves as a surrogate for these autonomic changes. Thus explaining why nonautonomically mediated changes in heart rate (as described above) do not improve outcomes. It is a paradigm shift to consider increasing the heart rate to attenuate sympathoexcitation.

Epidemiologic studies have demonstrated an elevated risk of sudden death related to exercise compared to sedentary periods (Whang et al. JAMA. 2006; 295: 1399, Albert et al. N Engl J. Med. 2000; 343: 1355, herein incorporated by reference in their entireties). The reported range for relative risk of sudden death related to exercise is very broad, apparently highly dependent on level of fitness and gender. The Nurse's Health Study (Whang et al. JAMA. 2006; 295: 1399, herein incorporated by reference in its entirety) found a relative risk of 2.38 related to moderate to vigorous exertion in almost 70,000 women, while the Physician's Health Study (et al. N Engl J. Med. 2000; 343: 1355, herein incorporated by reference in its entirety) found a relative risk of 16.9 related to vigorous exertion in approximately 21,000 male physicians. Pathophysiologic triggers for sudden death related to exercise include, for example, the development of myocardial ischemia, an increased risk of infarction due to mechanical factors resulting in plaque rupture, increased thrombogenicity, and increased risk for fatal ventricular arrhythmias. Supporting the role of ventricular tachyarrhythmias, it has been shown that implantable defibrillator shocks often occur in the setting of exertion or emotional states associated with sympathoexcitation.

Sympathetic stimulation increases (Miyazaki et al. Circulation. 1990; 82: 1008, herein incorporated by reference in its entirety) and parasympathetic stimulation decreases (Vanoli et al. Circ Res. 1991; 68: 1471, herein incorporated by reference in its entirety) the incidence of ventricular fibrillation in the setting of ischemia/reperfusion or infarction. The exercise and post-exercise recovery period are associated with sympathetic stimulation and parasympathetic withdrawal indicating that these changes could be implicated in the pathogenesis of sudden death due to ventricular tachyarrhythmias at this time. Furthermore, these changes in autonomic tone have effects on cardiac electrophysiology (Kannankeril et al. Am J Physiol Heart Circ Physiol. 2002; 282: H2091, herein incorporated by reference in its entirety).

The Determinants of Myocardial Infarction Onset Study examined the relative risks of myocardial infarction, a common precipitant of sudden cardiac death, in one hour increments for five consecutive hours after a bout of heavy physical exertion (Mittleman et al. Cardiol Clin. 1996; 14: 263, herein incorporated by reference in its entirety). Individuals were at greater susceptibility for only the one hour immediately after exertion. The time required for the heart rate to return to baseline values after moderate exercise has not been well characterized. Although the period of time for which an individual is at risk for sudden death following exercise has not been clearly defined, it is clear that it extends for a period of time following exercise. Experiments performed during development of embodiments of the present invention demonstrate that sympathoexcitation persists for at least 45 minutes after even brief (16 minutes) moderate exercise. Attenuating the sympathoexcitation in the recovery period is an important therapeutic target.

The vascular endothelium plays a pivotal role in the acute and chronic regulation of vessel wall homeostasis, thrombosis, and inflammation by secreting an array of molecules, such as nitric oxide (NO), prostacyclin (PGI), and endothelin-1 (ET-1) that exert highly regulated vasoactive and biological effects (Vane et al. N Engl J. Med. 1990; 323: 27, herein incorporated by reference in its entirety). The activity of endothelial cells is modulated by shear stress, metabolic products, and autonomic discharge, which contribute to the maintenance of a baseline vasodilatory tone and anti-inflammatory and antithrombotic milieu. When perturbation of this complex balance occurs, the endothelium may undergo dramatic functional changes and rapidly reverse its phenotype to a vasoconstrictor, proinflammatory and prothrombotic state, commonly known as “endothelial dysfunction”, which may act as a substrate for the development of atherosclerosis and acute cardiovascular complications (Drexler. Prog Cardiovasc Dis. 1997; 39: 287, herein incorporated by reference in its entirety).

Flow-mediated dilatation (FMD) has emerged as a powerful investigational tool for the assessment of endothelial function in conduit vessels such as the brachial artery. This technique indirectly assesses NO bioactivity in vivo and has become one of the most common tests in vascular research. FMD correlates with endothelium-dependent dilator responses in the coronary circulation (Matsuo et al. J Cardiovas Pharmacol. 2004; 44: 164, herein incorporated by reference in its entirety), but has the advantage of being non-invasive. Therefore, it is suitable for repeated testing also at short time intervals, thus allowing detection of acute effects of various interventions (Liang et al. Clin Sci (Loud). 1998; 95: 669, Campia et al. Am J Physiol Heart Circ Physiol. 2004; 286: H76, herein incorporated by reference in their entireties).

Most cardiovascular risk factors have been associated with reduced FMD, indicating that impaired NO-dependent vasodilation is a common pathway in the pathogenesis of vascular damage (Ghiadoni et al. Circulation. 2000; 102: 2473, Iiyama et al. Am Heart J. 1996; 132: 779, herein incorporated by reference in their entireties). Appropriate treatment of the disease may result in reversal of the abnormal vasodilatory response (Muiesan et al. Hypertension. 1999; 33: 575, herein incorporated by reference in its entirety). Many of these risk factors are associated with enhanced sympathetic activity, indicating a contribution of the sympathetic nervous system to the pathogenesis of endothelial dysfunction (Katz et al. J Am Coll Cardiol. 1992; 19: 918, Greenwood et al. Circulation. 1999; 100: 1305, herein incorporated by reference in their entireties).

Data indicate that sympathetic nervous system activation may impair FMD also in healthy subjects, suggesting a possible substrate for acute cardiovascular events independent of traditional risk factors (Etsuda et al. Clin Cardiol. 1999; 22: 417, herein incorporated by reference in its entirety). FMD of the brachial artery was measured before and during sympathetic stimulation achieved by baroreceptor unloading with a lower body negative pressure box. FMD responses were significantly attenuated by sympathetic stimulation, whereas no changes in endothelium-independent responses to nitroglycerin infusion (Hijmering et al. J Am Coll Cardiol. 2002; 39: 683, herein incorporated by reference in its entirety). FMD responses were studied under four different conditions of enhanced sympathetic tone: lower body suction, cold pressor test, mental arithmetic test (MAT), and activation of muscle chemoreflex. Blunting of FMD during the cold pressor test was observed, but not during lower body suction, mental arithmetic test, or muscle chemoreflex, indicating that blunted FMD may not be a general response to increased sympathetic activity (Dyson et al. Am J Physiol Heart Circ Physiol. 2006; 290: H1446, herein incorporated by reference in its entirety). Transient impairment of FMD in healthy middle-aged men during mental stress indicates that age may affect the relationship between sympathetic activation and FMD (Ghiadoni et al. Circulation. 2000; 102: 2473, herein incorporated by reference in its entirety). Acute exercise may negatively affect FMD (Harris et al. Obesity (Silver Spring). 2008; 16: 578, herein incorporated by reference in its entirety).

In some embodiments, the present invention comprises a method for suppressing sympathoexcitation in a subject. In some embodiments, cardiac pacing is administered to a subject (e.g. following exercise). In some embodiments, cardiac pacing comprises increasing a subject's heart rate over the subject's resting or baseline heart rate.

In some embodiments, the present invention comprises an implantable sensor (e.g. vibration sensor, accelerometers, dP/dt sensor, temperature sensor, respiration sensor, etc) that when activated raises the bradycardia pacing rate from the nominal pacing rate programmed for rest conditions to a higher pacing rate.

In some embodiments, the present invention comprises a defibrillator and/or pacemaker device configured to elevate a subject's heart rate following strenuous activity (e.g. exercise). In some embodiments, a device of the present invention comprises a heart rate sensor component (e.g. vibration sensor, accelerometers, dP/dt sensor, temperature sensor, respiration sensor, etc) and a defibrillator component. In some embodiments, a device of the present invention comprises a heart rate sensor component (e.g. vibration sensor, accelerometers, dP/dt sensor, temperature sensor, respiration sensor, etc) and a pacemaker component.

### EXPERIMENTAL

The following examples are provided in order to demonstrate and further illustrate certain preferred embodiments and aspects of the present invention and are not to be construed as limiting the scope thereof.

### Example 1

### Atropine Studies

In experiments performed during development of embodiments of the present invention, the parasympathetic effects on cardiac electrophysiology during the post-exercise recovery period were studied. Subjects underwent upright bicycle exercise testing on two occasions. Continuous 12-lead ECG monitoring was performed using a commercially available system (Quest Exercise Stress System, Burdick, Deerfield Wis.) for a baseline period of 5 minutes prior to exercise. Subjects underwent a 16-minute submaximal exercise protocol on a stationary bicycle with an initial work load of 50 Watts followed by increases to 75 Watts at 4 minutes and 100 Watts at 8 minutes, as tolerated by the patient. Continuous ECG was recorded for up to 45 minutes of recovery. Blood samples for determination of plasma epinephrine and norepinephrine samples were taken during the rest period, at minutes 8 and 15 of exercise, and during the recovery period at 5, 10, 20, 30, and 45 minutes after cessation of exercise. On the second day, the same exercise protocol was repeated, but intravenous atropine (0.04 mg/kg) was administered in four divided doses (0.01 mg/kg every 30 seconds) beginning at minute 12 of exercise. With regard to the changes in autonomic effects (and heart rate) there were no significant differences between the normal controls and those with coronary artery disease.

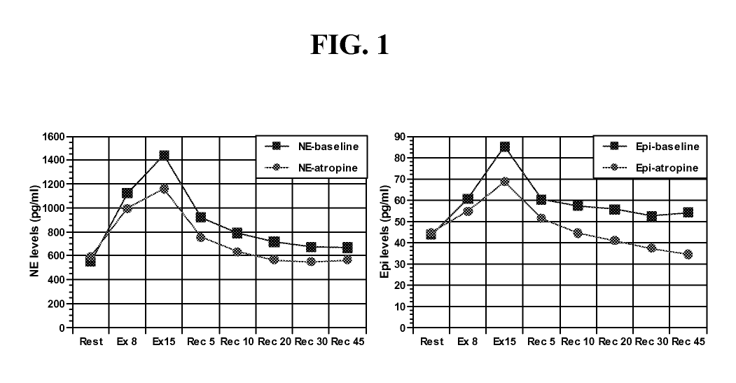

Plasma catecholamine levels (NE-norepinephrine; Epi-epinephrine) during the baseline exercise test and during the test in which atropine was administered during exercise (SEE FIG. 1). The baseline rest norepinephrine level was 547 pg/ml. As expected, it increased during exercise and declined during recovery. However, even 45 minutes after cessation of exercise, the plasma norepinephrine level remained significantly higher than at rest (668 pg/ml). Similar changes can be seen for plasma epinephrine on the right panel; specifically, 45 minutes after cessation of exercise, the plasma epinephrine level is also significantly higher than at rest. After atropine is administered during exercise, there are significant reductions in both the plasma norepinephrine and epinephrine levels. By 10 minutes of recovery, both the plasma norepinephrine and epinephrine levels returned to the resting values.

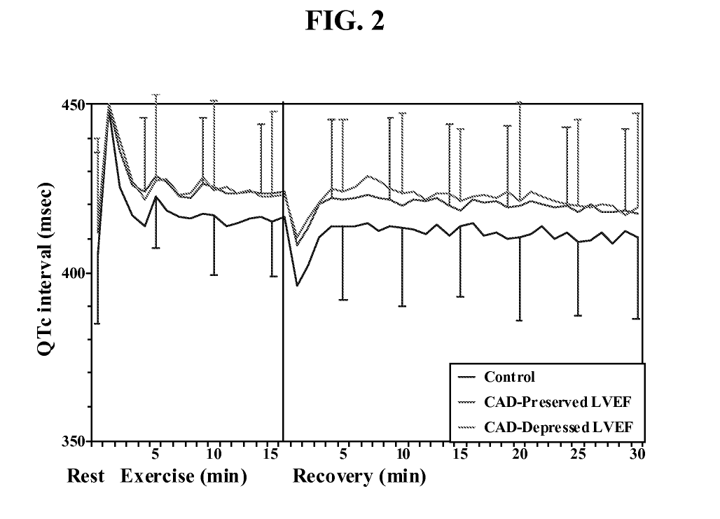

Accompanying the persistent sympathoexcitation was a persistent prolongation of the corrected QT interval (QTc). The controls had a shorter QTc than the subjects with coronary artery disease (CAD) with either preserved or depressed left ventricular ejection fraction (LVEF) at rest, during exercise, and during recovery (SEE FIG. 2). Throughout the recovery period, the QTc is 5-10 ms longer than at baseline.

Atropine is a muscarinic receptor blocker. Muscarinic blockade has been shown to increase norepinephrine release in the setting of nicotinic agonists (Lindmar et al. Br J Pharmac Chemother. 1968; 32: 280, herein incorporated by reference in its entirety). It is unlikely that atropine, a muscarinic blocker, exerts a direct effect to decrease plasma catecholamine levels. Thus, the observed reduction in plasma catecholamines is related to reflex inhibition due to a change in a physiologic parameter. The changes are most likely mediated by the carotid artery and aortic arch baroreceptors, which are involved in important feedback loops that regulate the feedback loop between heart rate and blood pressure. Activation of these receptors results in parasympathetic activation and sympathetic inhibition. The baroreceptors respond to the absolute (mean and peak) pressure and the rate of pressure change. Furthermore, with increasing exercise intensity, the baroreflex response is continuously reset rightward and upward. The manner in which all the changes in hemodynamic parameters during the recovery phase of exercise interact to form the feedback loops that govern sympathetic and parasympathetic activation/inhibition is complex. Atropine could have multiple effects on baroreceptor activation. For example, parasympathetic blockade could have direct or indirect effects on blood pressure, effects on contractility, and effects on heart rate. The direct effects of atropine on blood pressure and contractility are small, particularly in comparison to the large effects on heart rate. The heart rate and blood pressures (BP) during peak exercise and at various times in recovery are shown in the tables below during the baseline exercise test and during the exercise test in which atropine was administered during exercise:

| | Peak | Recovery | Recovery | Recovery | Recovery |
|---|---|---|---|---|---|
| | exercise | 5 min | 10 min | 20 min | 30 min |
| HEART | | | | | |
| RATE | | | | | |
| (bpm) | | | | | |
| Baseline | 115 ± 15 | 81 ± 12 | 78 ± 12 | 76 ± 11 | 74 ± 11 |
| Atropine | 133 ± 17 | 110 ± 16 | 106 ± 16 | 102 ± 15 | 99 ± 14 |
| Systolic BP | | | | | |
| (mmHg) | | | | | |
| Baseline | 162 ± 25 | 125 ± 14 | 121 ± 15 | 118 ± 18 | 118 ± 15 |
| Atropine | 162 ± 25 | 122 ± 15 | 120 ± 16 | 118 ± 14 | 117 ± 14 |
| Diastolic | | | | | |
| BP | | | | | |
| (mmHg) | | | | | |
| Baseline | 83 ± 12 | 77 ± 9 | 77 ± 9 | 75 ± 9 | 77 ± 10 |
| Atropine | 83 ± 14 | 78 ± 9 | 78 ± 9 | 78 ± 11 | 79 ± 9 |

There were large changes in heart rate with atropine, but no detectable changes in either systolic or diastolic blood pressure, thus indicating that the reduction in sympathoexcitation (to baseline values) associated with atropine is related to the accompanying acceleration in heart rate. This may be mediated by effects on mean pressure (e.g. shortening of the diastolic interval will increase the mean pressure) or by other responses to increased heart rate (e.g. effects due to increased cardiac output or contractility).

### Example 2

### Pacing Studies

Subjects with chronically implanted dual chamber pacemakers or defibrillators are recruited from the pacemaker database of Northwestern University Medical Center Subjects are only be enrolled if they participate in regular exercise and can maintain bicycle exercise for the duration of the studies. Subjects who cannot attain a heart rate of at least 100 beats/minute during exercise are excluded from evaluation. Subjects who cannot maintain one to one AV conduction at rest, during exercise, and during recovery are excluded. Subjects with active exertional angina are not enrolled so that this does not confound the results. Subjects with diabetes mellitus or known autonomic disorders are not studied. Subjects with clinical or electrocardiographic evidence of myocardial ischemia, unstable angina, or recent myocardial infarction (within the preceding three months) are excluded. Subjects with decompensated congestive heart failure within the preceding three months are excluded. Subjects with significant arrhythmias, such as atrial fibrillation or frequent ectopy are excluded. All subjects are screened for any cardiac complaints (chest pain, shortness of breath, palpitations, dyspnea on exertion). Subjects' medical regimens are not altered, but they are required to have been on a stable medical regimen for three months. Subjects unable to perform bicycle exercise are excluded; only subjects who participate in regular aerobic exercise a minimum of 60 minutes per week are studied. Highly trained endurance athletes are not studied. Subjects who cannot complete the exercise regimen on the first day for any reason are excluded.

All evaluations are performed on an outpatient basis. A full history and physical examination are performed for all subjects, including assessment of cardiac symptoms, detailed cardiac history, medication use, indication for device therapy, and assessment of physical activity (approximate minutes per week). Prior to the protocol exercise testing, all subjects undergo a symptom limited cardiopulmonary exercise stress test using the Bruce protocol to determine maximal exercise capacity. Standard 12-lead electrocardiograms and systolic and diastolic blood pressures, measured using a standard cuff sphygmomanometer, are obtained at rest, at the end of each stage of exercise and during the recovery period. Breath-by-breath respiratory gas analysis for measurement of oxygen and carbon dioxide are obtained on-line at rest, throughout exercise and during the recovery period, using a Medical Graphics Corporation Cardio-2 metabolic exercise cart (Minneapolis, Minn.). The subject's rating of perceived exertion (RPE) on the Borg Scale and respiratory exchange ratio is recorded at the end of each stage and at peak exercise. The peak exercise VO2 value is defined as the highest VO2 value achieved at end-exercise after reaching anaerobic threshold. The VO2 at anaerobic threshold values are determined as the point at which expired carbon dioxide increases in a nonlinear fashion relative to the rate of oxygen consumption by the V-slope method. The VO2 at anaerobic threshold value for each subject is visually noted by an independent cardiopulmonary exercise-testing specialist. The RPE, peak exercise VO2, and exercise duration is recorded.

All subjects undergo seated bicycle exercise testing on two separate days separated by at least 72 hours. A peripheral intravenous line is inserted into the forearm for blood draws and/or drug administration. Subjects are then attached to a cardiac monitor and ECG machine (Burdick Quest Exercise Stress System, Cardiac Science, Bothell, Wash.). Subjects are also attached to the Thoracic Electrical Bioimpedance Cardiac Output (TEBCO®, Hemo Sapiens Inc, Sedona, Ariz.) device for measurement of continuous cardiac output using the thoracic impedance method. Prior to exercise, a 5 mL blood sample is drawn through the indwelling catheter for measurement of electrolytes, BUN, glucose and creatinine levels. Plasma catecholamine levels are drawn at rest after assuming a seated resting position on the exercise bicycle for at least 5 minutes. All measurements are made with subjects seated on an electrically braked bicycle ergometer (SciFit ProII, Tulsa, Okla.). At the first session, a baseline 12-lead electrocardiogram and blood pressure is recorded at rest after assuming a seated position for at least 5 minutes. Continuous 12-lead ECG monitoring is performed for a 5 minute rest period. Following the rest period, brachial artery reactivity testing is performed. When completed, continuous 12-lead electrocardiogram monitoring is performed for a 16 minute exercise period and a 30 minute recovery period. Subjects will be instructed to maintain a pedal speed of approximately 80 revolutions per minute. The exercise protocol begins with a work load of 50 Watts, followed by an increase to 75 Watts at 4 minutes and 100 Watts at 6 minutes, if tolerated by the subject. Subjects continue to exercise at this workload for an additional 10 minutes. At the end of 16 minutes of exercise, the peak heart rate and blood pressure are recorded, and exercise will be stopped. Repeat blood pressures are obtained every 5 minutes for the remainder of the 30 minute recovery period. Catecholamine levels are drawn from the peripheral intravenous line at rest, at minutes 8 and 15 of exercise, and at minutes 5, 10, 20, and 30 of the recovery period. At 30 minutes of recovery, brachial artery reactivity testing are performed.

At the second session, the identical set-up and exercise protocol is performed. Blood draws for plasma catecholamines and brachial artery reactivity testing are performed at the same times. However, prior to the onset of exercise, the heart rate is temporarily increased to 100 beats/min by programming atrial pacing at this rate or at the rate pre-determined to be used for pacing during the recovery period. After one minute, a 12 lead ECG is obtained for comparison purposes to the paced ECGs in the recovery period. The pacing intervention begins at 2 minutes of recovery. Atrial pacing is activated via the subject's pacemaker or implantable defibrillator at 100 beats/min; this heart rate was chosen as it approximates the mean heart rate noted during recovery after atropine administration (see heart rate table above) and is faster than the intrinsic heart rate of most subjects (see histograms below). If the heart rate after 5 minutes of recovery on the first day was noted to be faster than 95 beats/min, the heart rate is set approximately 10 beats/min faster than the intrinsic heart rate determined during the initial test. Pacing is maintained at a minimum of 100 beats/min throughout the recovery period. The vast majority of subjects have a heart rate <90 beats/min by five minutes of recovery. The five minute heart rate on the first exercise day determines the intervention pacing rate. If the five minute heart rate on the first exercise day is ≦95 beats/min, the heart rate during recovery on the second day is programmed at 100 beats/min. If the five minute heart rate on the first exercise day is >95 beats/min, the heart rate during recovery on the second day is programmed approximately 10 beats/min faster (in 5 beat/min increments). Subjects whose heart rate does not recover to ≦110 beats/min by 5 min of recovery are not studied on the second day. After the test, the pacemaker is restored to its original parameters and heart rate and blood pressure are recorded.

Blood samples are collected in heparinized tubes and placed on ice. After centrifugation at 4° Celsius (C) at 3000 rpm for 15 minutes, 2 mL of plasma is transferred to an empty tube and stored at −70° C. for subsequent analysis.

ECG data is analyzed with custom software using the MATLAB program (Mathworks, Natick, Mass.). QRS detection is performed using a template matching algorithm. First, median templates of the QRS complexes are generated from a 10-second segment for each of the ECG leads using a slope based detection algorithm with the point of maximum negative slope chosen as the fiducial point. The cross-correlations of the templates with their respective signals are then summed across all leads and the QRS complexes are detected by finding the peaks of the resulting signal that exceeds a third of the maximum value. After identifying premature atrial and ventricular beats, the RR interval preceding the premature beat and the two RR intervals following the premature beat are excluded from further analysis.

QRS onset is identified using slope threshold criteria. T wave offset is estimated as the point where the tangent of the maximum descending T wave slope intersects the isoelectric line. The median values of all leads are chosen as the global QRS onsets and T wave offsets for each beat. An eleven beat median filter is used to filter out remaining QRS onset and T wave offset outliers. All automated computerized measurements are visually confirmed by two individuals. QT intervals are calculated. Corrected QT intervals (QTc) are obtained using Bazett's formula. Because each subject will have QT intervals measured at the same rate at rest and during recovery, rate independent comparisons will be performed using the raw QT interval values.

Endothelium-dependent (flow-mediated dilation, FMD) and -independent (nitroglycerin-mediated dilation) vasodilator function of the brachial artery is assessed (Campia et al. Am J Physiol Heart Circ Physiol. 2004; 286: H76, herein incorporated by reference in its entirety).

FMD is assessed on each study day prior to and 30 minutes post exercise. The sympathoexcitatory effects of exercise on endothelial function are determined by comparing FMD before and after exercise. Given the prolonged effects of nitroglycerin on brachial artery diameter, evaluation of endothelium-independent dilation is performed only post-exercise. Previous investigations have indicated that sympathetic activation affects FMD independently of changes in nitroglycerin-mediated dilation (Thijssen et al. Am J Physiol Heart Circ Physiol. 2006; 291: H3122, herein incorporated by reference in its entirety). Variations in FMD reflect the selective influence of sympathoexcitation on endothelial function. The effects of atrial pacing on endothelium-dependent and independent dilation are ascertained by comparing FMD and nitroglycerin-mediated dilation recorded after exercise on day 1 and day 2.

During the resting period, subjects are connected to a continuous ECG monitor and a pressure cuff is applied around the left upper arm. After a 10 minute rest period, the left brachial artery is visualized on the anterior aspect of the arm, 2-15 cm proximal to the antecubital fossa, with a high-resolution (>7.5 MHz) ultrasound probe. The position of the transducer is marked to retrieve the same portion of the artery in the post exercise study. After baseline images and flow measurements are obtained, the pressure cuff applied on the arm is inflated at 200-250 mmHg for 5 min. Blood flow is measured during the first 15 seconds after cuff deflation, and arterial image acquisitions for diameter measurements are performed between 45 and 90 s after cuff deflation. Post-exercise, endothelium-independent dilation of the brachial artery are assessed as follows: after at least 15 min of rest following cuff release, new baseline images and flow measurements are obtained, and 0.4 mg of nitroglycerin spray is given sublingually. Then, blood flow and brachial artery images are recorded 3 to 5 minutes after nitroglycerin administration. Arterial diameter is measured from the interface between vascular lumen and the endothelial layer at end diastole, incident with the R wave on the ECG. Images are analyzed by in a blinded fashion.

The following table summarizes the testing during Rest, Exercise, and Recovery (at the indicated times):

| | Rest | Exercise | Recovery |
|---|---|---|---|
| ECG - heart rate and | Continuous | Continuous | Continuous |
| QT interval | | | |
| Blood pressure | Rest | Peak | Every 5 min |
| Cardiac output | Continuous | Continuous | Continuous |
| Plasma | Rest | 8 and 15 min | 5, 10, 20, and 30 min |
| catecholamines | | | |
| Brachial artery | Rest | | 30 min |
| reactivity testing | | | |

Experiments conducted during development of embodiments of the present invention were used for sample size calculations and are presented in tables 1 and 2. As noted above, subjects were studied on two separate days. The first exercise test is referred to as the Baseline test. During the second exercise test, atropine was administered during exercise and is referred to as the Atropine test.

TABLE 1
N, Mean, and Standard Deviation for ln(NE) by
Atropine and Time in Recovery

| Measurement | N | Mean | SD |
|---|---|---|---|
| Baseline-Rest | 91 | 6.31 | 0.44 |
| Baseline-30^{th} min Recovery | 79 | 6.51 | 0.43 |
| Atropine-Rest | 90 | 6.38 | 0.42 |
| Atropine-30^{th} min Recovery | 81 | 6.31 | 0.40 |

TABLE 2
Variance-Covariance Matrix by Atropine and Time in Recovery-
bold numbers on the diagonal represent standard deviations,
while off-diagonal numbers are correlations.

| | | Baseline- | | |
|---|---|---|---|---|
| | Baseline- | Recovery | Atropine- | Atropine- |
| Measurements | Rest | 30 min | Rest | Rocovery 30 min |
| Baseline-Rest | 0.44 | | | |
| Baseline-30^{th} min | 0.70 | 0.43 | | |
| Recovery | | | | |
| Atropine-Rest | 0.81 | 0.68 | 0.42 | |
| Atropine-30^{th} min | 0.63 | 0.77 | 0.79 | 0.40 |
| Recovery | | | | |

Ln(NE) levels at the 30th minute in recovery (6.51±0.43) are 3.2% higher than they were at rest prior to exercise (6.31±0.44). Experiments indicate that pacing will reduce the disparity between resting and recovery ln(NE) levels by at least 75%. Therefore, ln(NE) levels at 30th minute in recovery during pacing stage will be 6.36±0.40. Some reduction in sample variability of ln(NE) levels during recovery after Atropine administration was observed, indicating that pacing will similarly reduce sample variability of the outcomes.

Table 2 shows that sample correlation between baseline and 30th minute outcome levels was 0.70 during baseline and 0.79 after Atropine. Recovery levels of ln(NE) follow baseline measurements more closely when subjects were administered Atropine, indicating that pacing will have similar effects. Resting catecholamine levels during Baseline and Atropine exercise tests were highly correlated. The correlation between 30th minute of recovery during Baseline and Atropine exercise tests was also very high. The correlations between resting catecholamine levels and 30th minute of recovery in the opposing exercise test study were relatively high but lower than those for the corresponding stage.

The variability of the outcome at all four measurement occasions was increased to the maximum level observed. Variability of the outcome levels for the recovery during pacing was set half-way between the observed value and maximum variability. All correlations were reduced while preserving the pattern of correlations observed in the prior study. These estimates were used in the final sample size estimation (table 3). The estimated sample size required to attain 90% power at the 5% two-tailed significance level was 76.

TABLE 3
Variance-Covariance Matrix by Pacing and Time in Recovery-
bold numbers on the diagonal represent means ± standard deviations;
off-diagonal numbers represent correlations.

| | | Baseline- | | Pacing- |
|---|---|---|---|---|
| | Baseline- | Recovery | Pacing- | Recovery |
| Measurements | Rest | 30 min | Rest | 30 min |
| Baseline - | 6.31 ± 0.44 | | | |
| Rest | | | | |
| Baseline - 30^{th} | 0.63 | 6.51 ± 0.44 | | |
| minute | | | | |
| Recovery | | | | |
| Pacing - Rest | 0.75 | 0.5 | 6.31 ± 0.44 | |
| Pacing - 30^{th} | 0.55 | 0.6 | 0.65 | 6.36 ± 0.42 |
| minute | | | | |
| Recovery | | | | |

The primary analysis of the catecholamine data will utilize the mixed effects model with random effects of subjects, pacing, and time in the study. Variance-covariance structure for the random effects will be selected with the help of likelihood ratio comparisons for nested models. Pacing (baseline vs. pacing) and Time as well as their multiplicative interaction will be used as fixed effects in the mixed effects model.

A limited amount of missing blood catecholamine measurements are likely to occur during the study. Such missing data are accommodated by the mixed effects analysis and the procedure uses all available information when maximizing the maximum likelihood on the fit to the data. Therefore, no imputation or list-wise deletion of the cases with missing data will be necessary. Conservative assumptions used in sample size calculations will be robust to the presence of the missing data. Similar analyses will be performed for the QT interval data and the brachial artery reactivity testing.

Distributions of the outcome variable during all study stages will be assessed using both statistical and graphical methods to validate the assumption about data normality. Natural logarithm transformation produces satisfactory approximation of the normal distribution; however, the assumption will be validated on the collected data. Alternative transformations will be entertained as well as non-parametric data analysis methods.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

The following references are herein incorporated by reference in their entireties:
1. Whang W, et al. Physical Exertion, Exercise, and Sudden Cardiac Death in Women. JAMA. 2006; 295: 1399.
2. Albert C, et al. Triggering of SD from Cardiac Causes by Vigorous Exertion. N Engl J. Med. 2000; 343: 1355.
3. Lind L, et al. The Effects of Mental Stress and the Cold Pressure Test on Flow-Mediated Vasodilation. Blood Press. 2002; 11: 22.
4. Hijmering M L, et al. Sympathetic Activation Markedly Reduces Endothelium-Dependent, Flow-Mediated Vasodilation. J Am Coll Cardiol. 2002; 39: 683.
5. Joras M, et al. The Association of Acute Exercise-Induced Ischaemia with Systemic Vasodilator Function in Patients with Peripheral Arterial Disease. Vasc Med. 2008; 13: 255.
6. Lindmar R, et al. A Muscarinic Mechanism Inhibiting the Release of Noradrenaline from Peripheral Adrenergic Nerve Fibres by Nicotinic Agents. Br J Pharmac Chemother. 1968; 32: 280.
7. Lahiri M K, et al. Assessment of Autonomic Function in Cardiovascular Disease: Physiological Basis and Prognostic Implications. J Am Coll Cardiol. 2008; 51: 1725.
8. Fletcher G F, et al. Statement on Exercise: Benefits and Recommendations for Physical Activity Programs for All Americans. American Heart Association. Circulation. 1996; 94: 857.
9. Jouven X, et al. Heart-Rate Profile During Exercise as a Predictor of SD. N Engl J. Med. 2005; 352: 1951.
10. Kjekshus J K. Importance of Heart Rate in Determining Beta-Blocker Efficacy in Acute and Long-Term Acute Myocardial Infarction Intervention Trials. Am J. Cardiol. 1986; 57: 43F.
11. Lechat P, et al. Heart Rate and Cardiac Rhythm Relationships with Bisoprolol Benefit in Chronic Heart Failure in CIBIS II Trial. Circulation. 2001; 103: 1428.
12. Gullestad L, et al. What Resting Heart Rate Should One Aim for When Treating Patients with Heart Failure with a Beta-Blocker? Experiences from MERIT-HF. J Am Coll Cardiol. 2005; 45: 252.
13. Miyazaki T, et al. Pericardial Prostaglandin Biosynthesis Prevents the Increased Incidence of Reperfusion-Induced VF Produced by Efferent Sympathetic Stimulation in Dogs. Circulation. 1990; 82: 1008.
14. Vanoli E, et al. Vagal Stimulation and Prevention of Sudden Death in Conscious Dogs with a Healed Myocardial Infarction. Circ Res. 1991; 68: 1471.
15. Kannankeril P J, et al. Parasympathetic Effects on Cardiac Electrophysiology During Exercise and Recovery. Am J Physiol Heart Circ Physiol. 2002; 282: H2091.
16. Mittleman M, et al. Physical Exertion as a Trigger of Myocardial Infarction and Sudden Cardiac Death. Cardiol Clin. 1996; 14: 263.
17. Vane J R, et al. Regulatory Functions of the Vascular Endothelium. N Engl J. Med. 1990; 323: 27.
18. Drexler H. Endothelial Dysfunction: Clinical Implications. Prog Cardiovasc Dis. 1997; 39: 287.
19. Matsuo S, et al. The Relationship between Flow-Mediated Brachial Artery Vasodilation and Coronary Vasomotor Responses to Bradykinin. J Cardiovas Pharmacol. 2004; 44: 164.
20. Liang Y L, et al. Non-Invasive Measurements of Arterial Structure and Function: Repeatability, Interrelationships and Trial Sample Size. Clin Sci (Loud). 1998; 95: 669.
21. Campia U, et al. Insulin Impairs Endothelium-Dependent Vasodilation Independent of Insulin Sensitivity or Lipid Profile. Am J Physiol Heart Circ Physiol. 2004; 286: H76.
22. Ghiadoni L, et al. Mental Stress Induces Transient Endothelial Dysfunction. Circulation. 2000; 102: 2473.
23. Iiyama K, et al. Impaired Endothelial Function with Essential Hypertension Assessed by Ultrasonography. Am Heart J. 1996; 132: 779.
24. Muiesan M L, et al. Effect of Treatment on Flow-Dependent Vasodilation of the Brachial Artery in Essential Hypertension. Hypertension. 1999; 33: 575.
25. Katz S D, et al. Impaired Endothelium-Mediated Vasodilation in the Peripheral Vasculature of Patients with Congestive Heart Failure. J Am Coll Cardiol. 1992; 19: 918.
26. Greenwood J P, et al. Single-Unit Sympathetic Discharge: Quantitative Assessment in Human Hypertensive Disease. Circulation. 1999; 100: 1305.
27. Etsuda H, et al. Morning Attenuation of Endothelium-Dependent, Flow-Mediated Dilation in Healthy Young Men: Possible Connection to Morning Peak of Cardiac Events? Clin Cardiol. 1999; 22: 417.
28. Dyson K S, et al. Effect of Acute Sympathetic Nervous System Activation on Flow-Mediated Dilation of Brachial Artery. Am J Physiol Heart Circ Physiol. 2006; 290: H1446.
29. Harris R A, et al. The Flow-Mediated Dilation Response to Acute Exercise in Overweight Active and Inactive Men. Obesity (Silver Spring). 2008; 16: 578.
30. Thijssen D H, et al. Sympathetic Nervous System Contributes to the Age-Related Impairment of Flow-Mediated Dilation of the Superficial Femoral Artery. Am J Physiol Heart Circ Physiol. 2006; 291: H3122.

We claim:

1. A method of suppressing sympathoexcitation in a subject comprising administering cardiac pacing to said subject.

2. The method of claim 1, wherein said sympathoexcitation comprises post-exercise sympathoexcitation.

3. The method of claim 1, wherein said cardiac pacing comprises and increased heart rate over said subject's resting heart rate.

4. The method of claim 1, wherein said cardiac pacing is administered by a defibrillator or pacemaker.

5. The method of claim 4, wherein said defibrillator comprises a cardioverter defibrillator.

6. The method of claim 1, wherein said suppressing sympathoexcitation reduces said subject's risk of ventricular tachyarrhythmia.

7. The method of claim 1, wherein said suppressing sympathoexcitation reduces said subject's risk of sudden cardiac death.

8. A device comprising a sensor component and a heart rate regulation component, wherein activation of said sensor component causes said heart rate regulation component to raise a subject's bradycardia pacing rate.

9. The device of claim 8, wherein raising said bradycardia pacing rate comprises raising said rate from the nominal pacing at rate rest conditions to a higher pacing rate.

10. The device of claim 8, wherein said device is implantable.

11. The device of claim 8, wherein raising said bradycardia pacing rate suppresses sympathoexcitation in said subject.

12. The device of claim 8, wherein raising said bradycardia pacing rate reduces said subject's risk of ventricular tachyarrhythmia

13. The device of claim 8, wherein raising said bradycardia pacing rate reduces said subject's risk of sudden cardiac death.

14. The device of claim 8, wherein said heart rate regulation component comprises a pacemaker or defibrillator.
